(19) United States
(12) Patent Application Publication
(10) Pub. No.: US 2022/0265393 A1
(43) Pub. Date: Aug. 25, 2022

(54) SYSTEMS AND METHODS FOR ORTHODONTIC TREATMENT INTERVENTION

(71) Applicant: Smylio Inc., Fremont, CA (US)

(72) Inventors: Loc Phan, Santa Clara, CA (US); Renjith Menon, Campbell, CA (US); Henry Hanh Chan, San Jose, CA (US)

(21) Appl. No.: 17/634,877

(22) PCT Filed: Aug. 10, 2020
(86) PCT No.: PCT/US20/45650

§ 371 Date: Feb. 11, 2022

Related U.S. Application Data
(60) Provisional application No. 62/885,633, filed on Aug. 12, 2019.
(60) Provisional application No. 63/046,839, filed on Jul. 1, 2020.

(51) Int. Cl. A61C 7/00; A61C 7/08; G16H 30/40; G16H 80/00

(52) U.S. Cl. CPC A61C 7/002; A61C 7/08; G16H 30/40; G16H 80/00

(57) ABSTRACT

Orthodontic intervention is performed after a patient, undergoing an orthodontic treatment plan utilizing orthodontic aligners, sends an image of their teeth remotely to an image processing module. The image is analyzed to determine current teeth position and crate an electronic model of the current teeth position. From the teeth position model, it is determined that the current teeth position is not compatible with the orthodontic treatment plan, which typically requires restarting the treatment plan and having the patient rescanned. Here, an electronic model of a rescue appliance is generated, where the rescue aligner is configured to move the teeth to a position back into compatibility with the orthodontic aligners. The rescue aligner is manufactured and sent to the patient without requiring the patient to visit any point of care.

### CROSS-REFERENCE TO RELATED APPLICATIONS

This application is a national stage application pursuant to 35 U.S.C. § 371 of International Application No. PCT/US20/45650, filed Aug. 10, 2020, which claims the benefit of U.S. Provisional Application No. 62/885,633, filed Aug. 12, 2019, and U.S. Provisional Application No. 63/046,839, filed Jul. 1, 2020. This application incorporates the aforementioned references herein.

### FIELD OF THE INVENTION

The subject matter of the present disclosure relates generally to the field of orthodontic devices. More particularly, the present disclosure relates to user removable orthodontic devices.

### BACKGROUND

An objective of orthodontics is to move a patient's teeth to positions where function and/or aesthetics are optimized. Traditionally, appliances such as braces are applied to a patient's teeth by a treating practitioner and the set of braces exerts continual force on the teeth and gradually urges them toward their intended positions. Over time and with a series of clinical visits and reactive adjustments to the braces by the practitioner, the appliances to move the teeth toward their final destination.

More recently, alternatives to conventional orthodontic treatment with traditional affixed appliances (e.g., braces) have become available. For example, systems including a series of molded plastic aligners have become commercially available from Align Technology, Inc., San Jose, Calif., under the trade name Invisalign® System. The Invisalign® System is described in numerous patents and patent applications assigned to Align Technology, Inc. including, for example in U.S. Pat. Nos. 6,450,807, and 5,975,893.

The Invisalign® System typically includes designing and fabricating multiple aligners to be worn by the patient before the aligners are administered to the patient and used to reposition the teeth (e.g., at the outset of treatment). Often, designing and planning a customized treatment for a patient makes use of computer-based 3-dimensional planning/design tools. The design of the aligners relies on computer modeling of the patient's teeth in a series of planned successive tooth arrangements, and the individual aligners are designed to be worn over the teeth, such that each aligner exerts force on the teeth and elastically repositions the teeth to each of the planned tooth arrangements.

Arguably, such aligners are less noticeable than traditional braces because typically aligners are constructed from a transparent material, however, many believe that aligners are easily noticeable due to the glossy sheen of the transparent material. Like traditional braces, aligners are required to be worn nearly constantly (20-22 hours a day), with breaks allowed for eating and cleaning teeth.

After fitting the patient an providing the patient with a series of aligners, follow-up visits are required to ensure that the orthodontic alignment plan is on track. The visits are required because the care provider needs to physically see the patient to assess the state of tooth alignment and perhaps perform quantitative or qualitative tests. Such visits can often be perfunctory because many alignment plans do not require modification, and as such add a heavy burden on patient's schedules and budgets.

For some patients, their teeth position goes out of the bounds of the prescribed treatment program and therefore becomes incompatible with the previously made set of aligners. In such cases, the patient is typically required to revisit the care provider to receive a new dental scan and later, receive a new set of aligners based on that scan. This can essentially restart the entire orthodontic aligner procedure for the patient, resulting in added expense and frustration. This is complicated by global events, for example, the global pandemic related to the coronavirus disease 2019 (COVID-19) has made point of visits an impossibility.

### SUMMARY OF THE INVENTION

Embodiments of the invention relate to orthodontic appliances, systems, and methods of use as summarized in the following paragraphs. Some embodiments relate to orthodontic methods for tracking and correcting tooth alignment without requiring the patient to visit a care provider.

Some embodiments relate to a method for performing orthodontic intervention, where at least one image of a patient's teeth can be received from a patient undergoing an orthodontic treatment plan utilizing a plurality of orthodontic aligners. The at least one image can be used to create a current teeth position model. It can be determined that the current teeth position model is not compatible with the plurality of orthodontic aligners. An electronic model can be generated of a rescue appliance configured to move the teeth to a position compatible with at least one aligner of the plurality of orthodontic aligners. The electronic model of the rescue appliance can be sent to an aligner manufacturer. The rescue appliance can be manufactured based on the electronic model of the rescue appliance. The rescue appliance can be sent from the aligner manufacturer directly to the patient.

In some embodiments, the at least one image can be electronically transmitted from a network access device of the patient and the at least one image file can be generated by the access device of the patient.

In some embodiments, the at least one image comprises a 2D image taken with a communication device of the patient and wherein the current teeth position model comprises a 3D model derived from the at least one 2D image.

In some embodiments, the current teeth position model can be generated by user observations and/or measurements of the at least one image.

In some embodiments, the current teeth position model is located outside an elastic working range of a current aligner of the plurality of aligners, the current aligner being configured for positioning the patient's teeth to a planned teeth position according to the orthodontic treatment plan.

In some embodiments, the rescue appliance can have a greater elastic working range than the elastic working range of the current aligner.

In some embodiments, the elastic working range of the rescue appliance can be 1.5-3 times greater than the elastic working range of the current aligner.

In some embodiments, the electronic model of the rescue appliance can be generated without requiring a rescan of the patient's teeth.

In some embodiments, the sending of the electronic model of the rescue appliance to the aligner manufacturer can be triggered by an electronic approval message sent by a care provider.

In some embodiments, the care provider can provide the electronic approval message after conducting a remote examination of the patient via the access device of the patient.

Some embodiments relate to a system for performing orthodontic intervention, where the system can include an image processing module, which can be configured to receive at least one electronic image of a patient's teeth from a patient undergoing an orthodontic treatment plan utilizing a plurality of orthodontic aligners. The image processing module can be configured to create a current teeth position model based on the at least one image. The system can include a rescue appliance model generation module, which can be configured to create an electronic model of a rescue appliance for moving the patient's teeth to a position compatible with at least one aligner of the plurality of orthodontic aligners without having to rescan the patient's teeth.

In some embodiments, the at least one image file can be electronically transmitted to the image processing module from a network access device of the patient and the at least one image file can be generated by the access device of the patient.

In some embodiments, the image processing module can be further configured to determine whether the current teeth position model is compatible with the plurality of orthodontic aligners.

In some embodiments, creation of the electronic model of the rescue appliance can be triggered by a determination that the current teeth position model is not compatible with the plurality of orthodontic aligners.

In some embodiments, the system can include an aligner manufacturing module that can be configured to receive the electronic model of the rescue appliance and initiate physical manufacture of the rescue appliance.

In some embodiments, the rescue appliance can be directly shipped to the patient after manufacture of the rescue appliance.

In some embodiments, the rescue appliance can have greater elastic working range than the elastic working range of any of the plurality of orthodontic aligners.

In some embodiments, the elastic working range of the rescue appliance can be 1.5-3 times greater than the elastic working range of any of the plurality of orthodontic aligners.

In some embodiments, the rescue appliance model generation module can be configured to send the electronic model of the rescue appliance to the aligner manufacturer after receiving an electronic approval message sent by a care provider network access device.

In some embodiments, wherein the electronic approval message can be provided after conducting a remote examination of the patient via the access device of the patient.

Some embodiments are related to a method for determining tooth position.

Some embodiments are related to a non-transitory processor-readable medium of an image analysis module, the non-transitory processor-readable medium can have processor-readable instructions configured to cause one or more processors of the image analysis device to perform a method for determining tooth position.

Some embodiments are related to an image analysis module having at least one processor. The at least one processor can be configured to perform a method for determining tooth position.

In some embodiments, at least one image file of a patient's teeth can be received from a patient undergoing orthodontic alignment.

In some embodiments, the at least one image file can be processed to create a tooth position model.

In some embodiments, a comparison of the tooth position model against a planned tooth position model can be created.

In some embodiments, the at least one image file can be created by an application of a mobile device of the patient.

In some embodiments, the at least one image file can be a video file.

In some embodiments, the at least one image file can be a plurality of image files.

In some embodiments, the at least one image file can be an image of an orthodontic appliance being worn by the patient.

In some embodiments, processing the at least one image file to create a tooth position model can include converting at least one 2D image into a 3D image.

In some embodiments, processing the at least one image file to create a tooth position model can include identifying areas of an orthodontic appliance worn by the patent in the at least one image file to determine areas of stress.

In some embodiments, the areas of stress can be identified according to different photographic qualities in comparison to portions of the orthodontic appliance undergoing relatively less stress.

In some embodiments, processing the at least one image file can include identifying at least one physical indicator on an orthodontic appliance worn by the patient in the image and using the at least one physical indicator to create at least one measurable aspects of the tooth position model.

In some embodiments, the at least one physical indicator can be a printed mark, indentation, or raised portion of the orthodontic appliance.

In some embodiments, the comparison includes a viewable report that can be electronically accessed by a care provider.

Some embodiments are related an orthodontic appliance that can have at least one shell shaped to receive teeth. The at least one shell can include at least one physical indicator, which can only useable for creating a tooth position model based on an image of orthodontic appliance.

### BRIEF DESCRIPTION OF THE DRAWINGS

For a better understanding of at least certain embodiments, reference will be made to the following Detailed Description, which is to be read in conjunction with the accompanying drawings.

FIG. 1 is a perspective view of a jaw and an orthodontic appliance, according to some embodiments.

FIG. 2 is an exploded view of an orthodontic appliance, according to some embodiments.

FIG. 3 is a connection schematic for an orthodontic appliance, according to some embodiments.

FIG. 4 is a perspective view of a process for molding an orthodontic appliance, according to some embodiments.

FIG. 5 is a schematic drawing of a network, according to some embodiments.

FIG. 6 is a screen shot of an application, according to some embodiments.

FIGS. 7A and 7B are flow diagrams of methods, according to some embodiments.

FIG. 8 is a schematic drawing of a computer system, according to some embodiments.

The figures depict various embodiments of the present invention for purposes of illustration only, wherein the figures use like reference numerals to identify like elements. One skilled in the art will readily recognize from the following discussion that alternative embodiments of the structures and methods illustrated in the figures may be employed without departing from the principles of the invention described herein.

### DETAILED DESCRIPTION

Embodiments are disclosed that can help a care provider remotely determine the condition of tooth alignment of a patient undergoing orthodontic treatment. In some embodiments, the patient can electronically transmit one or more images to an image processing device that the care provider can access. The one or more images can be taken via an application of the patient's mobile device, such as a smart phone or tablet. The image processing device can determine the state of tooth alignment based on analyzing the one or more images provided by the patient. Such embodiments can prevent perfunctory, in-person examinations of the patient by care providers.

Before the present invention is described in greater detail, it is to be understood that this invention is not limited to particular embodiments described, as such can, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which can be readily separated from or combined with the features of any of the other several embodiments without departing from the scope or spirit of the present invention. Any recited method can be carried out in the order of events recited or in any other order that is logically possible.

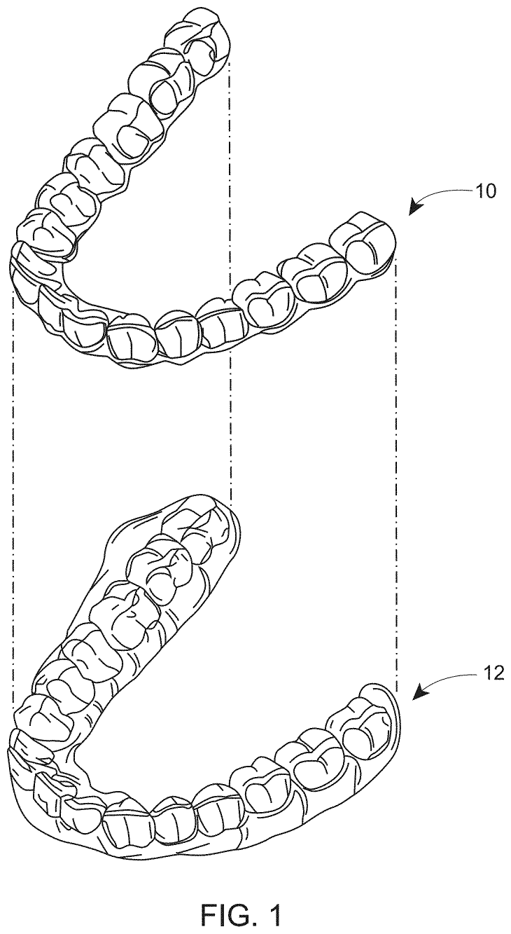

FIG. 1 provides an appropriate starting point in a detailed discussion of various embodiments of the present invention with respect to tooth repositioning appliances designed to apply repositioning forces to teeth. An orthodontic appliance 10 can be worn by a patient in order to achieve an incremental repositioning of individual teeth in the jaw 12. The orthodontic appliance 10 can include a shell having teeth-receiving cavities that receive and resiliently reposition the teeth. In some embodiments, a polymeric appliance can be formed from a sheet of suitable layers of polymeric material. An appliance can fit over all teeth present in an upper or lower jaw, or less than all of the teeth.

In some embodiments, only certain teeth received by an appliance will be repositioned by the appliance while other teeth can provide a base or anchor region for holding the appliance in place as it applies force against the tooth or teeth targeted for repositioning. In some cases, many or most, and even all, of the teeth will be repositioned at some point during treatment. Teeth that are moved can also serve as a base or anchor for holding the appliance as it is worn by the patient. Typically, no wires or other means will be provided for holding an appliance in place over the teeth. In some cases, however, it may be desirable or necessary to provide individual anchors on teeth with corresponding receptacles or apertures in the appliance so that the appliance can apply a selected force on the tooth. Basic methods for determining an orthodontic treatment plan using a series of incremented appliances as well as instructions for molding orthodontic appliances, are described in U.S. Pat. Nos. 8,512,037, 8,105,080, 7,245,750, 6,450,807, and 5,975,893, which are incorporated by reference herein, but only to an extent that those patents do not contradict the newer teachings disclosed herein.

An appliance can be designed and/or provided as part of a set of a plurality of appliances. In such an embodiment, each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. A target tooth arrangement can be a planned final tooth arrangement selected for the patient's teeth at the end of all planned orthodontic treatment. Alternatively, a target arrangement can be one of many intermediate arrangements for the patient's teeth during the course of orthodontic treatment. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

The orthodontic appliances can be generated all at the same stage or in sets or batches, e.g., at the beginning of a stage of the treatment, and the patient wears each appliance until the pressure of each appliance on the teeth can no longer be felt or has resulted in the maximum amount of expressed tooth movement for that given stage. A plurality of different appliances (e.g., set) can be designed and even fabricated prior to the patient wearing any appliance of the plurality. After wearing an appliance for an appropriate period of time, the patient replaces the current appliance with the next appliance in the series until no more appliances remain. The orthodontic appliances are generally not affixed to the teeth and the patient may place and replace the appliances at any time during the procedure (e.g., patient-removable appliances).

The final orthodontic appliance or several appliances in the series may have a geometry or geometries selected to overcorrect the tooth arrangement, i.e., have a geometry which would (if fully achieved) move individual teeth beyond the tooth arrangement which has been selected as the “final.” Such over-correction may be desirable in order to offset potential relapse after the repositioning method has been terminated, i.e., to permit movement of individual teeth back toward their pre-corrected positions. Over-correction may also be beneficial to speed the rate of correction, i.e., by having an appliance with a geometry that is positioned beyond a desired intermediate or final position, the individual teeth will be shifted toward the position at a greater rate. In such cases, the use of an appliance can be terminated before the teeth reach the positions defined by the appliance.

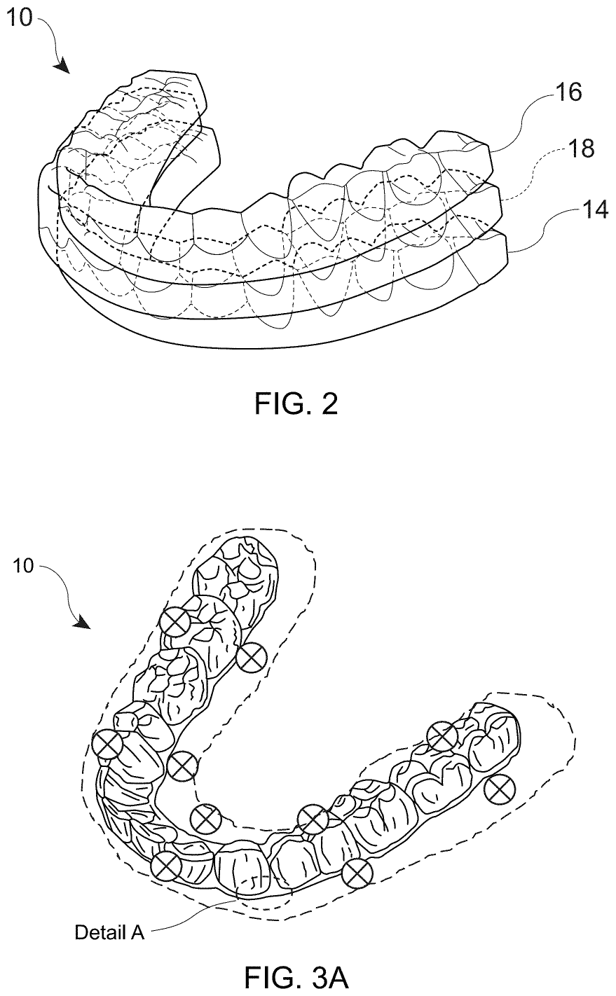

FIG. 2 shows an exploded view of an example of the orthodontic appliance 10. The orthodontic appliance 10 can include a first shell 14 having a teeth engaging surface and an opposite upper surface. The orthodontic appliance 10 can also include a second shell 16 having a lower-shell engaging surface and an opposite upper surface that is exposed to the mouth. Optionally, one or more additional shells 18 can be located between the first shell 14 and the second shell 16. In some embodiments, the more shells that are used, the greater the working elasticity of the orthodontic appliance 10, assuming use of the same material for each shell.

While the orthodontic appliance 10 is shown in an exploded view for the purpose of better understanding, in some embodiments, the shells of the orthodontic appliance 10 are intended to be mechanically engaged with one another in a stack. “Mechanically engaged” is defined herein as the substantially non-affixed or varyingly affixed engagement between one or more shells to approximate the strength of a single shell appliance of approximately the same thickness as the stacked shells. Mechanical engagement can be obtained by stacking the shells while having the lower-shell engaging surface of the second shell largely conforming to the upper surface of the first shell. In some embodiments, shells can be stacked loosely, i.e., without a compressive or an interference fit between shells or such that an upturned stack of shells self-disassembles, before being made substantially non-affixed or varyingly affixed. The shells are substantially non-affixed (or varyingly affixed) because a substantial amount of surface areas between the shells are not bonded or otherwise made inseparable through some process, with the remaining surfaces being affixed. In some embodiments, substantially non-affixed or varyingly affixed shells have less than 1-2%, 1-5%, 1-10%, 1-20%, 1-40%, 1-60%, or 1-80% of the combined contacting surfaces of the shells affixed. The area of non-fixation can be limited according to the needs of the appliance, hence, in some embodiments, a majority the surface areas of the appliance are affixed, while the remaining part is non-affixed because only the latter requires high working elasticity.

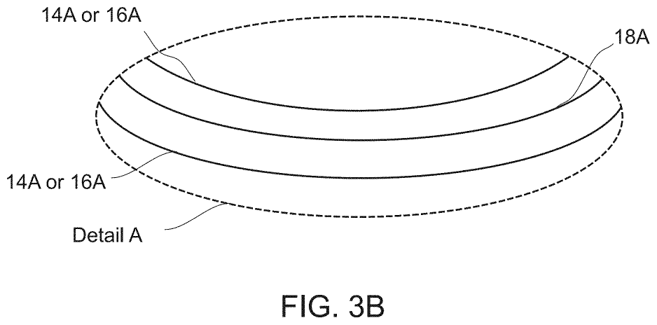

FIG. 3A shows a schematic for affixing the shells of the orthodontic appliance 10 at discrete locations. Each encircled “X” represents a possible point of fixation between the shells. Alternatively, as shown by the dashed line, the edges of each shell can serve as a continuous or non-continuous area of fixation. Generally, the more fixation provided, the less working elasticity the orthodontic appliance 10 will have. Points of fixation can be determined based on the amount of working elasticity required, which teeth are being moved, and which teeth are serving as anchors. Alternatively, the shells can be uniformly and weakly bonded with a highly elastic material of low cohesive strength that allows for a large amount of stretching and/or shearing. Such embodiments are substantially non-affixed or varyingly affixed because the working flexibility of such an orthodontic appliance are maintained due to the properties of the weak bond.

In some embodiments, shells of the orthodontic appliance 10 can be non-identical such that surface areas of one shell is greater or less than another shell. Accordingly, in some embodiments, edges, which are defined by the top and bottom surfaces of each shell, of such shells can be separated by gaps (e.g. 0.20-3.0 mm), as depicted by FIG. 3B, which shows an example with three shells 14, 16, 18 and three edges 14a, 16a, 18a. In some embodiments, referring to the arrangement shown at FIG. 2, bottom-most shell 14 can have the greatest surface area, resulting in edge 14a being at the bottom most position, shown, with shells 18 and 16 respectively having smaller surfaces areas such that edge 16a is at the top-most position. In such embodiments, the shells 14, 16, 18 are stacked such that steps formed by edges 14a, 16a, 18a face outward, away from the teeth. In some embodiments, referring to the arrangement shown at FIG. 2, top-most shell 16 can have the greatest surface area, resulting in edge 16a being at the bottom most position, shown, with shells 18 and 14 respectively having smaller surfaces areas such that edge 14a is at the top-most position. In such embodiments, the shells 14, 16, 18 are stacked such that inward facing steps formed by edges 14a, 16a, 18a face inward, i.e., towards the teeth.

Providing one or more of such gaps can be used to tune flexural modulus of the orthodontic appliance 10 and also result in less tongue irritation to the patient that can occur due to material thickness where edges are bonded at the same location. To alleviate irritation, gaps can be placed in areas that face inwards towards the mouth, resulting in stepped edges (e.g., edges 14a, 16a, 18a) facing the tongue, or the tooth-engaging shell can have a smaller surface area than shells stacked thereon, resulting in interior, tooth-facing steps and a single shell edge (e.g., edge 16a) that can contact the tongue. In some embodiments, the bottom-most, tooth-engaging shell, can have a greater or lesser total surface area than a second shell stacked thereon, which can result in at least a portion of the edge of the second shell being separated from the edge of the tooth-engaging shell. In some embodiments, only portions of the edges that face towards the mouth have such a gap, and in other embodiments, a uniform or non-uniform gap can exist between the entirety of edges. In some embodiments, the orthodontic appliance 10 can include shells, each having different surface areas.

The shells can have thicknesses ranging from 0.001-0.015 inches thick, and can be constructed from a polyester, a co-polyester, a polycarbonate, a thermoplastic polyurethane, a polypropylene, a polyethylene, a polypropylene and polyethylene copolymer, an acrylic, a cyclic block copolymer, a polyetheretherketone, a polyamide, a polyethylene terephthalate, a polybutylene terephthalate, a polyetherimide, a polyethersulfone, a polytrimethylene terephthalate or a combination thereof. In some embodiments, shells are coated with lubricous materials or provided with surface treatments to decrease friction between the shells. In some embodiments, interior portions of the shells are treated with hydrophobic coatings to prevent liquid intrusion into the shells. In some embodiments, shells of relatively more flexibility can be used in conjunction with stiffer shells. Flexible shells can be constructed from hydrogels, styrenic block copolymers (SBC), silicone rubbers, elastomeric alloys, thermoplastic elastomers (TPE), thermoplastic vulcanizate (TPV) elastomers, polyurethane elastomers, block copolymer elastomers, polyolefin blend elastomers, thermoplastic co-polyester elastomers, thermoplastic polyamide elastomers, or a combination thereof. Flexible shells may also provide the benefit of a gasket to prevent liquid intrusion between the shells.

In some embodiments, the lack of substantial fixation between shells provides greater working elasticity to the orthodontic appliance 10 because the teeth-engaging shell can flex more due to being thinner while the outer shells are allowed to flex in multiple directions away from the teeth-engaging shell. In some embodiments, this can result in partial mechanical disengagement between some of the engaging surfaces of the shells, however the disengagement is not enough to significantly impair flexural modulus of the device required for aligning the teeth to the target position.

In some embodiments, the shells of the orthodontic appliance 10 can be affixed to one another, for example, the orthodontic appliance 10 of FIG. 1 can be formed from a laminate material (e.g. fully bonded shells or a co-extruded layered material) or a single shell (i.e., only one of shell 14/16/18). In some embodiments, high working elasticity can be obtained by differing materials, material properties, and/or mechanical properties for the shells, which can also be used to further increase elasticity of the orthodontic appliance using non-affixed shells. For example, one shell, such as shell 18 can be formed from an elastomer material or a relatively high elastic polymer material as compared to the other shells of the orthodontic appliance 10. In some embodiments, high working elasticity can be obtained by differing material properties for one or more of the shells, for example, portions of one or more of the shells can have areas of cross-linked zones adjacent to non-crosslinked zones (e.g., localized crosslinking for a thermoplastic material by radiation cross-linking, chemical cross-linking with organic peroxides, or cross-linking using silane-grafting agents). In another example, portions of one or more of the shells can have areas with localized plasticizer doping (e.g. using esters, phthalates) to increase the elasticity of such portions. In some embodiments, high working elasticity can be obtained by differing mechanical properties for one or more of the shells, for example, portions of one or more of the shells can have areas of decreased wall thickness and/or areas of zero wall thickness (i.e., perforations). In some embodiments, perforations can be uniformly applied to a shell or only in certain locations, and be round, square, rectangular, and/or elongated.

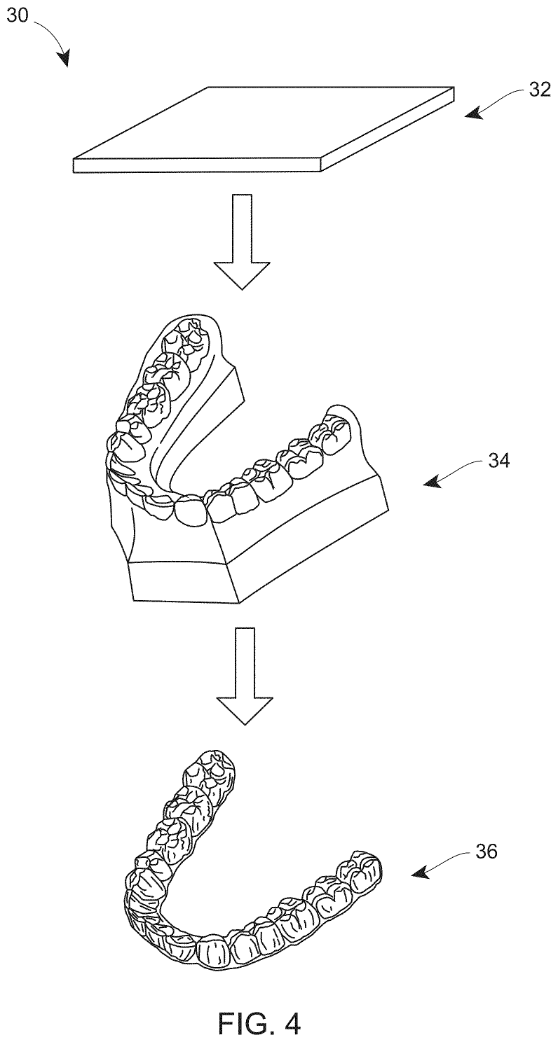

FIG. 4 depicts an example of a basic process 30 for forming an orthodontic appliance. As shown, a material 32 can be formed into an orthodontic appliance 36. The material 32 can be of one layer to form a single shell or multiple non-affixed layers of material to form multiple shells at once. In this example process, the tooth positioning appliance 36 can be produced with the use of a physical tooth model, or mold, 34. The tooth positioning appliance 36 can be produced by heating the thermoformable material 32 and then vacuum or pressure forming the material over the teeth in the physical tooth model 34. The tooth positioning appliance 36 is a direct representation of the physical tooth model. In some embodiments, material 32 is dimensioned (e.g., 120 mm and/or 125 mm diameter circle) for ready processing on a commercially available forming device (e.g., Erkoform®, Erkoform-3dmotion®, Biostar®, Ministar S®, Drufomat Scan®, Drufosmart®, Essix® SelectVac®). Guidelines for operating such forming devices can be found at Scheu Dental Technology, Biostar Operating Manual, DE/GB/FR/IT/ES/1.000/06/19 G REF PM 0113.01; Scheu Dental Technology, Application booklet for the pressure moulding technique, GB 2.000/07/19 G REF 0111.02; Erkodent, Thermoforming, S15-3106-48; Erkodent, Erkoform 3D, 61-8002-2; Erkodent, Erkoform-3D+ Instructions, BA-Erkoform-3d+-anl-EN-04-04-2019, which are incorporated by reference herein.

After formation, shells can be affixed to one another according to the desired working elasticity required for the patient. Methods of fixation include chemical bonding, localized melting, fasteners, and/or localized physical deformation to key the shells together. Before or after fixation takes place, excess material from the sheet can be trimmed to form a final tooth positioning appliance that can be used for orthodontic treatment of a patient. The edges of the shells can be sealed with a flexible material such as silicone to prevent liquid intrusion.

One or a series of physical tooth models, such as the model described above, may be used in the generation of elastic repositioning appliances for orthodontic treatment. Similar to the process above, each of the appliances can be generated by thermoforming a multilayer polymeric material over a mold of a desired tooth arrangement to form a dental appliance. The tooth positioning appliance of the desired tooth arrangement generally conforms to a patient's teeth but is slightly out of alignment with the initial tooth configuration. Placement of the elastic positioner over the teeth applies controlled forces in specific locations to gradually move the teeth into the desired configuration. Repetition of this process with successive appliances comprising new configurations eventually moves the teeth through a series of intermediate configurations to a final desired configuration.

In some embodiments, the orthodontic appliances disclosed herein can be used as rescue appliance, which is an orthodontic appliance configured to be used as an interceding appliance in an ongoing orthodontic therapy using a series of orthodontic appliances. For various reasons, such as patient non-compliance, at some point during the orthodontic therapy, the patient's teeth positioning may be too far out of position to continue with the therapy. In such cases, the prescribed orthodontic aligners do not have enough working elasticity to safely attach to the out of position teeth. When such events arise, the current standard of care requires the patient to have their teeth rescanned (via a dedicated tooth scanning apparatus) for a completely new set of aligners. One advantage of the rescue appliance is to bring the patient's teeth positioning back into compatibility with the original therapy plan and orthodontic aligners, and in some embodiments, this can be done without requiring the patient to be rescanned at a health care facility.

A rescue appliance can be configured in the manner of the orthodontic appliances disclosed herein, which have a much greater amount of working elasticity in comparison to standard orthodontic appliances. The elasticity of the orthodontic appliance 10 enables it to a greater elastic working range, i.e., the range of movement between a deformed position of the orthodontic appliance 10 (i.e., where the orthodontic appliance 10 is elastically deformed) and a non-deformed position. In some embodiments, the elastic working range of the orthodontic appliance is 1.5-3 times greater than the elastic working range of a prior art polymer aligner. This enables prescribing a single rescue appliance in such cases where patient tooth position has gone off track, i.e., where no previously prescribed aligner is elastically compatible with the current teeth position, with respect to the prescribed aligners, where ordinarily a completely new set of aligners would be required. For example, in a case where a patient's current teeth position (i.e., teeth position X) is not on track between a first prescribed aligner (i.e. prescribed teeth position A) and a second prescribed aligner (i.e. prescribed teeth position B), an orthodontic appliance 10 can be configured as a rescue appliance to bring the teeth position into a position of compatibility with the first aligner or the second aligner or a different previously prescribed aligner. In this manner, use of a rescue appliance curtails the requirement to have the patient be rescanned for a new set of aligners.

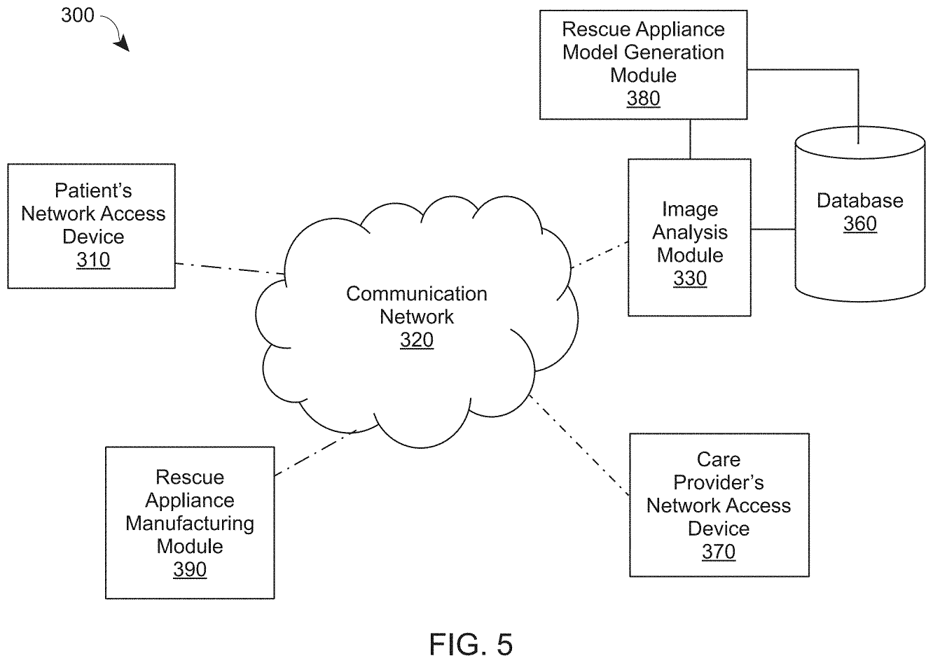

FIG. 5 illustrates an embodiment of network 300 for facilitating communication with a patient network access device 310. The network access device 310 can be a smart phone or tablet that is configured to communicate via a communication network 320. The network access device 310 can include a camera that is configured, via an application stored as processor executable instructions on a non-transitory medium readable by a processor of the network access device 310, to record one or more pictures of the patient's teeth and upload the pictures to an image analysis device 330. In some embodiments, the application can also be a platform for private electronic communications (e.g., encrypted text, email, voice, videoconferencing) with a health care provider using network access device 370, for the purpose of evaluating the patient's progress with an orthodontic treatment plan.

The network access device 310 can be configured by the application to enable a patient to take a photo of their teeth. As used herein, the terms (in singular or plural form) image, 2D photo, 2D picture, picture, or photo are defined to mean a single electronic image, plurality of electronic images, an electronic video, or a plurality of electronic videos. In some embodiments, the application can access the camera function of the network access device 310 and provide guidance to the patient for taking one or more pictures of the teeth. Guidance may be given, for example, by providing alignment features on the screen of the device so that the patient can provide one or more 2D pictures of the teeth according to specific camera angles, which can be based on providing triangulate images to determine relative depth. For example, a single face view of the teeth or multiple views of different views can be required. In some embodiments, pictures are taken with and/or without a worn appliance.

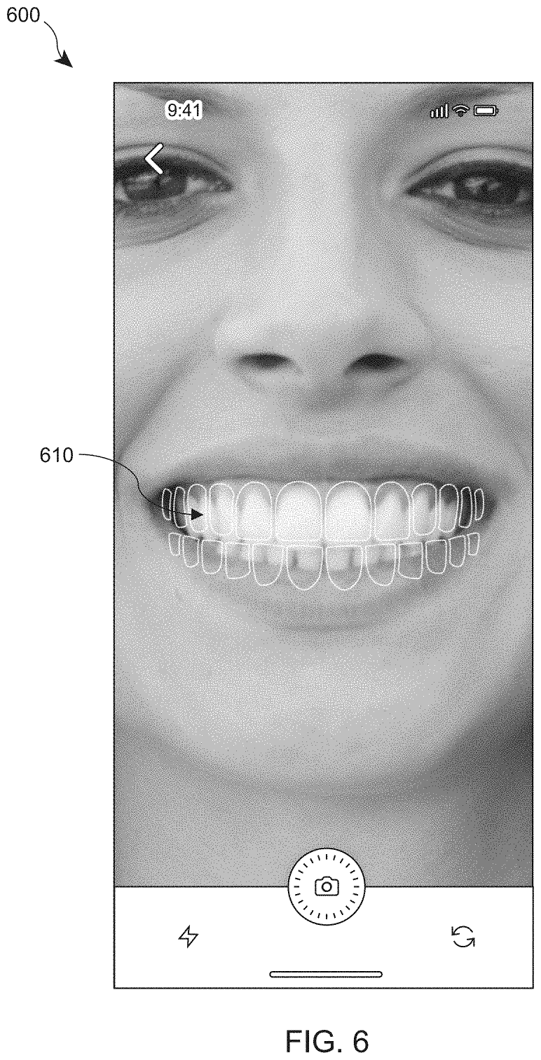

An example of such a guide is shown at FIG. 6, which shows a screen shot 600 of a tooth overlay 610 that the patient uses to align their own teeth on the screen. The overlay 610 can continuously appear on the screen of the network access device 310 while in a picture taking mode. In some embodiments, the overlay 610 can provide a visual indication (e.g. flash intermittently, change color) that the camera is correctly aligned. In some embodiments, the camera will take a picture when the overlay 610 is positioned correctly relative to the teeth without requiring further input from the patient to operate the camera.

The image analysis module 330 can be configured to analyze the pictures to determine the current position of one or more teeth of the patient. In some embodiments, the image analysis module 330 can be configured, via an application stored as processor executable instructions on a non-transitory medium readable by a processor of the image analysis module 330. In some embodiments, the image analysis module 330 can include software and/or hardware aspects of a server, special-purpose computer, or general-purpose computer, and communicatively coupled to a database 360.

In some embodiments, the image analysis module 330 can identify aspects captured on 2D pictures that can be used to determine position of teeth. In some embodiments, the image analysis module 330 can determine relative position of teeth based on a comparison of the 2D image to a planned position of the teeth, which can be derived from an orthodontic treatment protocol. This determination can be based on identifying and quantitatively measuring physical aspects captured in the 2D pictures.

In some embodiments, one or more 2D photos are of a patient's teeth while wearing an appliance and/or a patient's teeth while not wearing an appliance, and/or of an appliance not being worn. The appliance can be deformed by the teeth when worn, hence the worn appliance can be observed for attributes that show positional deviance from a planned treatment position.

In some embodiments, the 2D photo is processed to determine 3D attributes of the teeth. The 3D attributes can be used to compare with existing 3D models of the appliances. In some embodiments, the 3D attributes are used to create a comparison 3D model. Multiple triangulated images can be used to create a depth map in order to create a 3D model, such as a wire form model or a model with surfaces. In addition, known data from creating the appliances can be used to supplement the depth map. In some embodiments, physical indicators of the appliance can be used to create the depth map. Such aspects can be non-transparent or semi-transparent portions of the appliance that are easily differentiated from other surfaces of the appliance. Techniques for creating a 3D model from 2D images of teeth is described at U.S. patent Ser. No. 10/248,883, which is incorporated by reference herein.

The photos can include images of one or more indicators of the appliance. The indicators can be used to measure the location of the teeth based on quantitative measurements of attributes of the indicators. In some embodiments, the location of an indicator of a worn appliance can be directly compared to the location of an indicator of an unworn appliance. The difference (i.e., distance) between the locations can be quantitively measured and used to determine relative difference between the current positions of the teeth and the planned positions. The indicators can be for example, printed marks and/or ridges, bumps, or other physical attributes of the aligner.

In some embodiments, the stresses in the aligner material can be observed to determine relative position of teeth. When the aligner material is stressed, it can have different refractive indices when viewed through polarized light. In some embodiments, a polarized lens can be used to in conjunction with the network access device 310 to take photographs of the teeth. The amount of tooth movement toward a desired position can be correlated according to the amount of stress observed.

In some embodiments, the image analysis module 330 can prepare a report that estimates a degree of tooth movement relative to the desired tooth movement based on qualitative and/or quantitative analyses of the photos. The report can be based on comparison between the results of the photo analysis to 3D models created for the generation of the appliances. The report can provide information to a care provider in terms of estimates (e.g., tooth movement is X % on track) and/or provide a visual report with generated 3D models or other qualitative information.

In some embodiments, the image analysis module 330 can communicate with a rescue appliance model generation module (RAMGM) 380, which can be configured to process a 3D model generated by the image analysis module 330 for creating a rescue appliance. For example, in a case where a patient's current teeth position (i.e., teeth position X) is not on track between a first prescribed aligner (i.e. prescribed teeth position A) and a second prescribed aligner (i.e. prescribed teeth position B), a model of a rescue appliance, designed to bring the teeth position into a position of compatibility with the first aligner or the second aligner or a different previously prescribed aligner, can be generated by RAMGM 380. In some embodiments, RAMGM 380 can generate a model of a rescue appliance based on qualitative and/or quantitative data determined from user (e.g., an orthodontist or dental imaging technician) or software derived observations and/or measurements of the 2D pictures. In some embodiments, the RAMGM 380 can be configured, via an application stored as processor executable instructions on a non-transitory medium readable by a processor of the RAMGM 380. In some embodiments, RAMGM 380 can include software and/or hardware aspects of a server, special-purpose computer, or general-purpose computer, and communicatively coupled to a database 360.

A care provider using network access device 370 can access the records stored on database 360 by communicating with image analysis module 330 through communications network 320. The records can be assigned to a specific patient and formatted for use by a specific application of network access device 370, or alternatively accessible on records server 340 via a web-based application. Based on the records, a care provider can determine whether the treatment plan is on track or not on track, and in case of the later requires a physical inspection of the patient's teeth. In some embodiments, image analysis module 330 will trigger a communication to the network access device 370 when it is determined that a rescue appliance may be required to continue treatment of the patient. The communication can include a quantitative analysis of the current position of the patient's teeth, 3D model current position of the patient's teeth, comparative 3D model with respect to where the teeth should be positioned according to the orthodontic treatment plan, and/or a 3D model of a proposed rescue appliance to bring the teeth back into compliance with the orthodontic treatment plan.

The network access device 370 can also be used to privately communicate with the patient, via private electronic communications (e.g., encrypted text, email, voice, videoconferencing) with the patient's network access device 310, for the purpose of gathering information inputted by the patient in their patient's network access device 310, upload information (e.g. records, 3D tooth models) to the patient's network access device 310, and in some embodiments remotely conduct check-ups and examinations with the patient to determine how well the patient is proceeding with the orthodontic treatment plan, discuss compliance or pain issues with the patient, remotely view the patient's teeth, and discuss changes to the orthodontic treatment plan, such as implementation of a rescue appliance. In some embodiments, the care provider can approve of the model of the rescue appliance generated by RAMGM 380, for example after evaluating the patient remotely, and trigger a process whereby the rescue aligner model is manufactured and delivered to the patient.

A rescue appliance manufacturing module (RAMM) 390 can electronically communicate with the image analysis device 330 in order to receive the model of the rescue appliance. In some embodiments, this occurs after approval by the care provider. RAMM 390, or a manufacturing aspect in communication with RAMM 390, can then process the 3D model of the rescue appliance to create a mold or other manufacturing implement required to create the rescue appliance. In some embodiments, RAMM 390 can be configured, via an application stored as processor executable instructions on a non-transitory medium readable by a processor of the RAMM 390. In some embodiments, RAMM 390 can include software and/or hardware aspects of a server, special-purpose computer, or general-purpose computer, and communicatively coupled to manufacturing aspects, such as modules for controlling an aligner manufacturing apparatus. In some embodiments, the rescue appliance is manufactured according to the process of FIG. 4. After the rescue appliance is complete, it can be shipped directly to the patient for use.

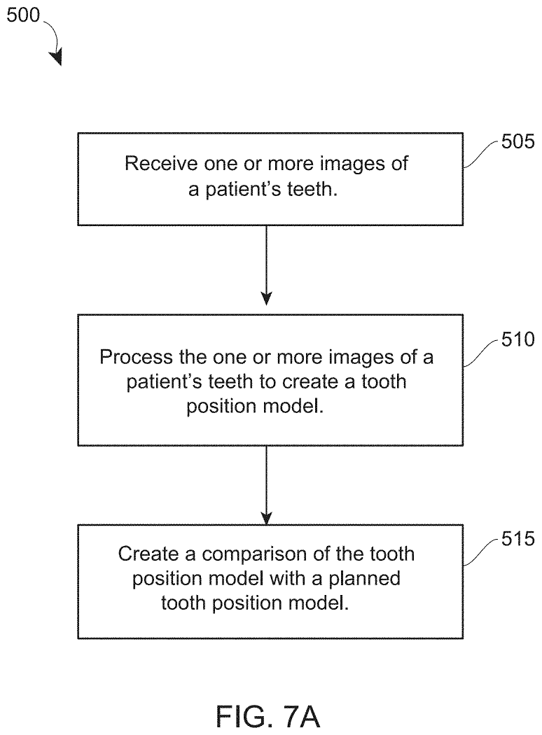

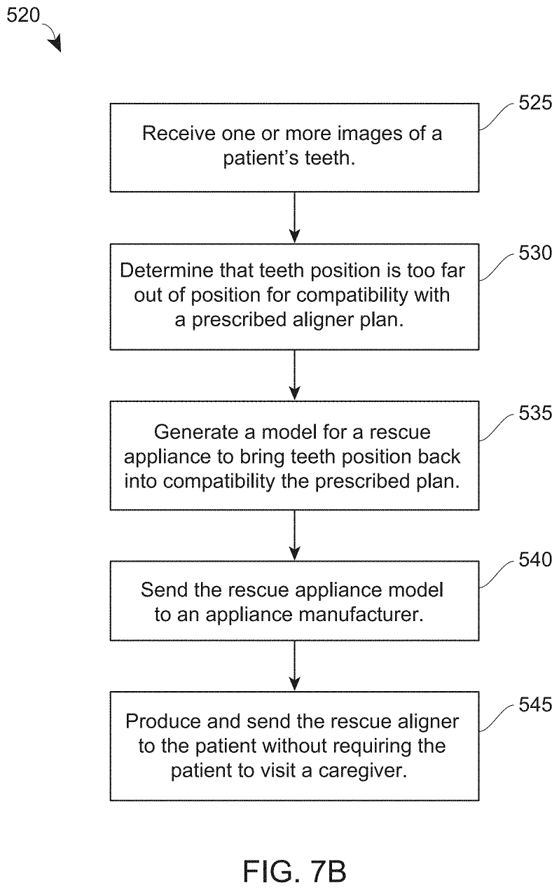

FIG. 7A illustrates method 500 that can be performed by at least one processor of a computing device, such as the image analysis device 330. Method 500 can be stored as processor executable instructions on a non-transitory medium readable by the processor. The processor can be configured to execute method 500.

At operation 505, the processor receives one or more image of a patient's teeth. The images can be electronic images that are taken by a personal device (e.g. network access device 310), such as a mobile phone or tablet, of the patient, or a device of a care provider, and electronically transmitted via a network to the processor. The images can be a videos or still images. The images can be taken with the assistance of alignment features on the screen of the personal device so that the patient can provide one or more 2D pictures of the teeth according to predetermined camera angles.

In some embodiments, the images can be one or more 2D photos of the patient's teeth while wearing an appliance and/or a patient's teeth while not wearing an appliance, and/or of an appliance not being worn. The appliance can be deformed by the teeth when worn, hence the worn appliance can be observed for attributes that show positional deviance from a planned treatment position.

At operation 510, the processor processes the images to create a tooth position model for determining positions of the teeth. In some embodiments, 2D images can be processed to form an outline or other simplified 2D model. In some embodiments, the process can include a 2D to 3D conversion of the images, to create a 3D image or wireframe model. Triangulated images can be used to create a depth map in order to create a 3D model, such as a wire form model or a model with surfaces. In addition, known data from creating the appliances can be used to supplement the depth map. In some embodiments, physical indicators of the appliance can be used to create the depth map. Such aspects can be non-transparent or semi-transparent portions of the appliance that are easily differentiated from other surfaces of the appliance.

In some embodiments, the process can include qualitative analysis of attributes of the images. Such analysis can include approximation of material stress of a photographed worn aligner. When the aligner material is stressed, it can have different refractive indices when viewed through polarized light. The stressed aligner material will thus have different photographic qualities than surrounding aligner material undergoing less stress. Such qualities may appear as darkened or lightened areas. In some embodiments, a polarized lens can be used to in conjunction with the patient's personal device to take photographs of the teeth. The amount of tooth movement toward a desired position can be correlated according to the amount of stress observed.

At operation 515, the processed images can be used to determine relative tooth position with respect to a planned tooth position model, which is based on an orthodontic plan to align the patient's teeth from a first position to a final position. This comparison can be used by a care provider to determine if the orthodontic therapy is on track or need revisiting. If the therapy is going according to plan, then unnecessary office visits for physical examinations can be avoided. In some embodiments, the location of an indicator of a worn appliance can be directly compared to the location of an indicator of an unworn appliance. The difference between the locations can be quantitively measured and used to determine relative difference between the current positions of the teeth and the planned positions.

In some embodiments, operation 515 includes preparation of an electronically viewable record or report that estimates a degree of tooth movement relative to the desired tooth movement based on qualitative and/or quantitative analyses of the photos. The report can be based on comparison between the results of the photo analysis to 3D models created for the generation of the appliances. The report can provide information to a care provider in terms of estimates (e.g., tooth movement is X % on track) and/or provide a visual report with generated 3D models or other qualitative information.

FIG. 7B illustrates method 520 that can be performed by system for generating a rescue appliance, such as the system shown at FIG. 5. In some embodiments, all or portions of method 520 can be stored as processor executable instructions on a non-transitory medium readable by one or more processors. The one or more processors can be configured to execute method 520.

At operation 520, the one or more processors receive one or more image of a patient's teeth undergoing an orthodontic treatment plan using a set of aligners. The images can be electronic images that are taken by a personal device (e.g. network access device 310), such as a mobile phone or tablet, of the patient, or a device of a care provider, and electronically transmitted via a network to the processor. The images can be a videos or still images. The images can be taken with the assistance of alignment features on the screen of the personal device so that the patient can provide one or more 2D pictures of the teeth according to predetermined camera angles. In some embodiments, the images can be one or more 2D photos of the patient's teeth while wearing an appliance and/or a patient's teeth while not wearing an appliance, and/or of an appliance not being worn. The appliance can be deformed by the teeth when worn, hence the worn appliance can be observed for attributes that show positional deviance from a planned treatment position. In some embodiments, a 3D model of the patient's current teeth position is generated from the 2D images.

At operation 530, the one or more processors determines that the patient's current teeth position is too far out of position for continued compatibility with the orthodontic treatment plan, i.e., the prescribed aligners will not be able to successfully fit the patient's current teeth position. In some embodiments, the determination can be made according to user inputs based on the user's qualitative and/or quantitative observations of the electronic 2D photos and/or the generated 3D model of the current position of the teeth. In some embodiments, the determination is made according to a process whereby the current position of the teeth is compared to the optimal and/or plan-compatible positions of the teeth that were determined for the patient according to their orthodontic treatment plan. For example, each aligner generated for the patient will have a limited elastic range of movement, and if the current position of the teeth is outside those ranges, then the processor can determine that the orthodontic treatment plan progress as originally planned. This determination can trigger generation of a rescue aligner to enable use of the patient's aligners.

At operation 535, the one or more processors can process the 3D model generated by the image analysis device 330 for creation of a rescue appliance model. For example, in a case where a patient's current teeth position (i.e., teeth position X) is not on track between a first prescribed aligner (i.e. prescribed teeth position A) and a second prescribed aligner (i.e. prescribed teeth position B), a model of a rescue appliance, designed to bring the teeth position into a position of compatibility with the first aligner or the second aligner or a different previously prescribed aligner, can be generated by the one or more processors. In some embodiments, the one or more processors can generate a model of a rescue appliance based on qualitative and/or quantitative data determined from user or software derived observations and measurements of the 2D pictures.

At operation 535, the one or more processors can send the model of the rescue appliance to an appliance manufacturer, which at operation 540 can in turn create a mold or some other manufacturing implement required to create the rescue appliance, form the rescue appliance, and send the rescue appliance to the patient for use. In some embodiments, the model of the rescue appliance is sent immediately upon its generation, and in other embodiments, a trigger is required to send the model to the manufacturer, such as an electronic authorization trigger sent from a care provider device. Advantageously, the generation of the patient's current teeth position, identification of the patient's incompatibility with the orthodontic treatment plan, generation of the model for the rescue appliance, and the physical generation of the rescue appliance can all take place without the patient visiting a point of care

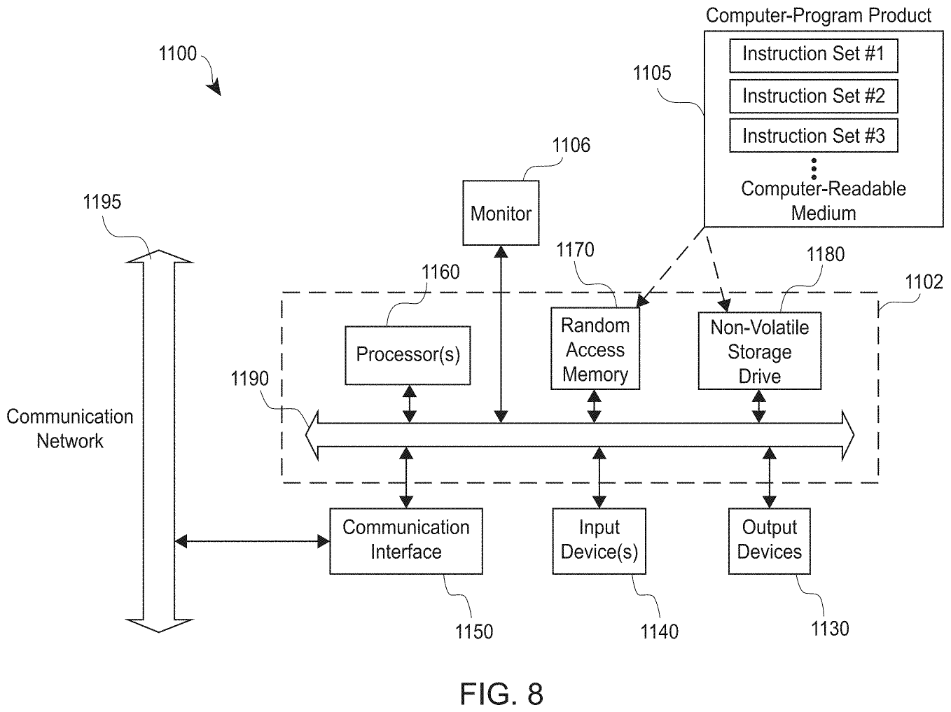

With reference to FIG. 8, an embodiment of a special-purpose computer system 1100 is shown. For example, one or more intelligent components, processing system 110 and components thereof may be a special-purpose computer system 1100. Such a special-purpose computer system 1100 may be incorporated as part of any of the other computerized devices discussed herein, such devices shown at FIG. 5. The above methods may be implemented by computer-program products that direct a computer system to perform the actions of the above-described methods and components. Each such computer-program product may comprise sets of instructions (codes) embodied on a computer-readable medium that direct the processor of a computer system to perform corresponding actions. The instructions may be configured to run in sequential order, or in parallel (such as under different processing threads), or in a combination thereof. After loading the computer-program products on a general-purpose computer system 1126, it can be transformed into the special-purpose computer system 1100.

Special-purpose computer system 1100 comprises a computer 1102, a monitor 1106 coupled to computer 1102, one or more additional user output devices 1130 (optional) coupled to computer 1102, one or more user input devices 1140 (e.g., keyboard, mouse, track ball, touch screen) coupled to computer 1102, an optional communications interface 1150 coupled to computer 1102, a computer-program product 1105 stored in a tangible computer-readable memory in computer 1102. Computer-program product 1105 directs computer system 1100 to perform the above-described methods. Computer 1102 may include one or more processors 1160 that communicate with a number of peripheral devices via a bus subsystem 1190. These peripheral devices may include user output device(s) 1130, user input device(s) 1140, communications interface 1150, and a storage subsystem, such as random-access memory (RAM) 1170 and non-volatile storage drive 1180 (e.g., disk drive, optical drive, solid state drive), which are forms of tangible computer-readable memory.

Computer-program product 1105 may be stored in non-volatile storage drive 1180 or another computer-readable medium accessible to computer 1102 and loaded into random access memory (RAM) 1170. Each processor 1160 may comprise a microprocessor, such as a microprocessor from Intel® or Advanced Micro Devices, Inc.®, or the like. To support computer-program product 1105, the computer 1102 runs an operating system that handles the communications of computer-program product 1105 with the above-noted components, as well as the communications between the above-noted components in support of the computer-program product 1105. Exemplary operating systems include Windows® or the like from Microsoft Corporation, Solaris® from Sun Microsystems, LINUX, UNIX, and the like.

User input devices 1140 include all possible types of devices and mechanisms to input information to computer 1102. These may include a keyboard, a keypad, a mouse, a scanner, a digital drawing pad, a touch screen incorporated into the display, audio input devices such as voice recognition systems, microphones, and other types of input devices. In various embodiments, user input devices 1140 are typically embodied as a computer mouse, a touch screen, camera, wireless remote, drawing tablet, or voice command system. User input devices 1140 can allow a user to select, input, or add objects, icons, text, photos, and the like that appear on the monitor 1106 via a command such as a click of a button or the like. User output devices 1130 include various types of devices to output information from computer 1102. These may include a display (e.g., monitor 1106), printers, non-visual displays such as audio output devices, etc.

Communications interface 1150 provides an interface to other communication networks, such as communication network 1195, and devices and may serve as an interface to receive data from and transmit data to other systems, WANs and/or the Internet. Embodiments of communications interface 1150 typically include an Ethernet card, a modem (telephone, satellite, cable, ISDN), a (asynchronous) digital subscriber line (DSL) unit, a USB interface, a wireless network adapter, and the like. For example, communications interface 1150 may be coupled to a computer network, or the like. In other embodiments, communications interface 1150 may be physically integrated on the motherboard of computer 1102, and/or may be a software program, or the like.

RAM 1170 and non-volatile storage drive 1180 are examples of tangible computer-readable media configured to store data such as computer-program product embodiments of the present invention, including executable computer code, human-readable code, or the like. Other types of tangible computer-readable media include floppy disks, removable hard disks, optical storage media such as CD-ROMs, DVDs, bar codes, semiconductor memories such as flash memories, read-only-memories (ROMs), battery-backed volatile memories, networked storage devices, and the like. RAM 1170 and non-volatile storage drive 1180 may be configured to store the basic programming and data constructs that provide the functionality of various embodiments of the present invention, as described above.

Software instruction sets that provide the functionality of the present invention may be stored in RAM 1170 and non-volatile storage drive 1180. These instruction sets or code may be executed by the processor(s) 1160. RAM 1170 and non-volatile storage drive 1180 may also provide a repository to store data and data structures used in accordance with the present invention. RAM 1170 and non-volatile storage drive 1180 may include a number of memories including a main random-access memory (RAM) to store instructions and data during program execution and a read-only memory (ROM) in which fixed instructions are stored. RAM 1170 and non-volatile storage drive 1180 may include a file storage subsystem providing persistent (non-volatile) storage of program and/or data files. RAM 1170 and non-volatile storage drive 1180 may also include removable storage systems, such as removable flash memory.

Bus subsystem 1190 provides a mechanism to allow the various components and subsystems of computer 1102 to communicate with each other as intended. Although bus subsystem 1190 is shown schematically as a single bus, alternative embodiments of the bus subsystem may utilize multiple busses or communication paths within the computer 1102.

Throughout the foregoing description, and for the purposes of explanation, numerous specific details are set forth in order to provide a thorough understanding of the described techniques. It will be apparent, however, to one skilled in the art that these techniques can be practiced without some of these specific details. Although various embodiments that incorporate these teachings have been shown and described in detail, those skilled in the art could readily devise many other varied embodiments or mechanisms to incorporate these techniques. Also, embodiments can include various operations as set forth above, fewer operations, or more operations; or operations in an order. Accordingly, the scope and spirit of the invention should be judged in terms of the claims, which follow as well as the legal equivalents thereof.

1. A method for performing orthodontic intervention, the method comprising:
receiving at least one image of a patient's teeth from a patient undergoing an orthodontic treatment plan utilizing a plurality of orthodontic aligners;
using the at least one image to create a current teeth position model;
determining that the current teeth position model is not compatible with the plurality of orthodontic aligners;
generating an electronic model of a rescue appliance configured to move the teeth to a position compatible with at least one aligner of the plurality of orthodontic aligners;
sending the electronic model of the rescue appliance to an aligner manufacturer; and
manufacturing the rescue appliance based on the electronic model of the rescue appliance;
and sending the rescue appliance from the aligner manufacturer directly to the patient.

2. The method of claim 1, wherein the at least one image is electronically transmitted from a network access device of the patient and wherein the at least one image file was generated by the access device of the patient.

3. The method of claim 1, wherein the at least one image comprises a 2D image taken with a communication device of the patient and wherein the current teeth position model comprises a 3D model derived from the at least one 2D image.

4. The method of claim 1, wherein the current teeth position model is generated by user observations and/or measurements of the at least one image.

5. The method of claim 1, wherein the current teeth position model is located outside of an elastic working range of a current aligner of the plurality of aligners, the current aligner being configured for positioning the patient's teeth to a planned teeth position according to the orthodontic treatment plan.

6. The method of claim 5, wherein the rescue appliance has greater elastic working range than the elastic working range of the current aligner.

7. The method of claim 6, wherein the elastic working range of the rescue appliance is 1.5-3 times greater than the elastic working range of the current aligner.

8. The method of claim 1, wherein the electronic model of the rescue appliance is generated without requiring a rescan of the patient's teeth.

9. The method of claim 1, wherein sending the electronic model of the rescue appliance to the aligner manufacturer is triggered by an electronic approval message sent by a care provider.

10. The method of claim 9, wherein the care provider provides the electronic approval message after conducting a remote examination of the patient via the access device of the patient.

11. A system for performing orthodontic intervention, the system comprising:
an image processing module configured to receive at least one electronic image of a patient's teeth from a patient undergoing an orthodontic treatment plan utilizing a plurality of orthodontic aligners and create a current teeth position model based on the at least one image;
a rescue appliance model generation module configured to create an electronic model of a rescue appliance for moving the patient's teeth to a position compatible with at least one aligner of the plurality of orthodontic aligners without having to rescan the patient's teeth.

12. The system of claim 11, wherein the at least one image file is electronically transmitted to the image processing module from a network access device of the patient and wherein the at least one image file was generated by the access device of the patient.

13. The system of claim 11, wherein the image processing module is further configured to determine whether the current teeth position model is compatible with the plurality of orthodontic aligners.

14. The system of claim 11, wherein creation of the electronic model of the rescue appliance is triggered by a determination that the current teeth position model is not compatible with the plurality of orthodontic aligners.

15. The system of claim 11, further comprising an aligner manufacturing module configured to receive the electronic model of the rescue appliance and initiate physical manufacture of the rescue appliance.

16. The system of claim 15, wherein the rescue appliance is shipped to the patient after manufacture of the rescue appliance.

17. The system of claim 11, wherein the rescue appliance has greater elastic working range than the elastic working range of the current aligner.

18. The system of claim 17, wherein the elastic working range of the rescue appliance is 1.5-3 times greater than the elastic working range of the current aligner.

19. The system of claim 11, wherein the rescue appliance model generation module is configured to send the electronic model of the rescue appliance to the aligner manufacturer after receiving an electronic approval message sent by a care provider network access device.

20. The system of claim 19, wherein the electronic approval message is provided after conducting a remote examination of the patient via the access device of the patient.
